(19) United States
(12) Patent Application Publication
(10) Pub. No.: US 2008/0294095 A1
(43) Pub. Date: Nov. 27, 2008

(54) OUTFLOW RATE REGULATOR

(76) Inventor: Jaime Zacharias, Santiago (CL)

Correspondence Address: JAIME ZACHARIAS, AV. LUIS PASTEUR 5917 - VITACURA, SANTIAGO, 6670775, CL

(21) Appl. No.: 11/754,284

(22) Filed: May 26, 2007

(51) Int. Cl. A61M 31/00

(52) U.S. Cl. 604/65

(57) ABSTRACT

An outflow rate regulator system for use in a phacoemulsification system to prevent the anterior chamber collapses that occur after occlusion breaks caused by fluid surges in the aspiration line. The outflow rate regulator system consisting in a flow limiting device installed in the aspiration line capable of varying the section or the extension of a fluid passage as a function of the pressure difference across the outflow rate regulator access and exit sides. The device is designed to reduce the outflow fluid passage area as a function of an increasing pressure difference across the outflow rate regulator. Alternatively, the effective extension of a narrow fluid passage is designed to increase as the pressure difference across the outflow rate regulator increases. Resistance to flow is increased with increasing pressure differences across the device in reversible manner. Clogging of the narrow fluid passages is avoided by upstream removal of solid particles above a determined size by a retaining filter.

### FIELD OF THE INVENTION

The present invention generally relates to a flow-rate control system and more particularly is related to an outflow rate control system for ophthalmic surgical equipment of the kind used for crystalline lens removal such as phacoemulsification equipment.

### BACKGROUND OF THE INVENTION

Typically, cataracts, or crystalline manifestations, in an eye are removed by fragmentation thereof which may include a hollow needle inserted into the eye through a small incision. Removal of the fragmented lens is effected through a centre hole in the needle and involves continuous circulation of fluid through the eye provided by positive pressure fluid irrigation and vacuum fluid aspiration which is provided to the hollow needle inserted therein. Ultrasound, water-jet, laser and other forms of energy can be transferred to the lens tissue by the hollow needle inserted in the eye to help fragment, disrupt and emulsify the cataract material to facilitate the removal of the crystalline lens fragments through the needle conduct together with the circulating fluid. Flow rate entering the aspiration line must be controlled to prevent excessive outflow that produces instability and collapse of the anterior chamber of the eye. This condition is particularly prone to occur after the breaking of occlusions that occur at the hollow needle tip by crystalline lens material. When an occlusion occurs, vacuum rises inside the aspiration system by the action of the aspiration pump located in the unit console. During vacuum rise a contraction occurs in the elastic walls of the aspiration system that is a function of the magnitude of the vacuum. Also, bubbles in the aspiration line will expand by the action of vacuum. Expansion of the contracted walls and contraction of the expanded bubbles when pressure drops creates a volume deficit that has to be filled by volume from the eye chamber. The release of the needle tip blockage allows fluid to travel from the anterior chamber of the eye towards the aspiration line at high flow rates because of the high pressure gradient created during occlusion. Compliance of the aspiration line will determine how fast and how much volume is needed to restore balance. Compliance will depend on rigidity of the walls of the aspiration line and the eventual presence of bubbles in the line. After occlusion break the outflow rate can overshoot to a flow rate that is higher than the console preset outflow rate value (above 60 cc/min peak). This peak in aspiration flow rate rapidly drops to the steady state outflow rate that is equal or lower that the console preset outflow rate depending on the outflow system resistance. Normally, the irrigation system is too slow to fully compensate he fluid void inside the eye chamber created by the outflow system peak suction. The current trend to reduce the incision size for lens removal procedures has further reduced the capabilities of the irrigation system to compensate post occlusion surges because of the increasing resistance to inflow at the incision level. This increases the chances of a negative fluid balance and a transient collapse of the anterior chamber of the eye that can lead to serious complications. The appearance and the magnitude of a post-occlusion surge will be determined by a series of factors such as infusion line pressure (irrigation bottle height), infusion resistance, aspiration line outflow rate, vacuum in the aspiration line at the moment of occlusion break, tubing material and structure, phacoemulsification needle tip resistance, presence of an aspiration bypass systems and eventual bubbles in the aspiration line. One way to reduce post-occlusion surge has been to increase irrigation bottle height but this condition over-pressurizes the eye with unknown consequences. Several active and passive post-occlusion surge reducing devices have been proposed to increase the vacuum level safely in order to remove the crystalline lens fragments with reduced amounts of energy. For example one passive device to reduce surge consists in coiling the outflow tubing to exponentially increase resistance as flow rate increases. This system increases the length of the tubing making it uncomfortable for the user. Another passive surge control system consists in a stricture in the aspiration line (i.e. 0.35 mm diameter port) that has high resistance to high flow rates (Cruise Control System, Staar, USA.). This system increases resistance and reduces maximum flow rate under non occlusion conditions affecting performance. Also, active post-occlusion surge limiting devices have been proposed usually based on feed-back loops that adjust flow rate or vent the aspiration line when an occlusion related state is detected to reduce the post-occlusion surge phenomenon. As an example, an aspiration line pressure sensing method and active flow control has been proposed for phacoemulsification systems in U.S. Pat. No. 5,392,653 entitled “Pressure transducer magnetically-coupled interface complementing minimal diaphragm movement during operation”. The above-referenced patent is incorporated herein by specific reference thereto. It is desirable to provide a surge control system that is inexpensive, simple, and does not affect performance of the lensenctomy system under non occlusion conditions.

### SUMMARY OF THE INVENTION

According to the principles of the present invention, an outflow rate regulator is provided for use with an ophthalmic surgical instrument having a hand-piece with a lens removing hollow needle in fluid communication with an aspiration line adapted to carry the fluid and particles of emulsified lens debris away from the surgical site. In accordance with one aspect of the present invention, the outflow rate regulator includes a flow limiting device adapted to be placed in fluid communication with the aspiration line that connects the aspiration pump and the hollow needle. The flow limiting device defines a fluid passage offering a variable resistance to flow that limits post occlusion surge in the anterior chamber of the eye following an occlusion break occurring at the distal portion of the aspiration line The fluid passage section of the outflow rate regulator is designed to vary resistance to flow across the device as a function of the difference in pressure between an access side and an exit side of the flow rate controlling device. The fluid passage can be acted upon to vary resistance to flow either by modifying the section of the fluid passage, by modifying the length of a narrow fluid passage or a combination of both as a function of the difference in pressure between an access side and an exit side of the flow rate controlling device. In this way increasing flow rates encounter a progressive resistance to flow produced by reduction of the fluid passage section or increased length produced by a mechanism that reacts to an increment in a pressure difference between an access and an exit side as sensed by a differential pressure sensor element. By varying several design aspects of the outflow rate regulator, different free flow-rate versus real flow-rate curves can be achieved that can better adapt to different real word surgical settings and instrumentation to prevent anterior chamber collapse caused by post occlusion surge. The free flow-rate versus real flow-rate function can deviate from linearity in several forms and can include hysteresis on purpose by variations in design. A single outflow rate regulator can incorporate an adjustment feature to program a desired performance of the device to accommodate to different surgical environments. This adjustment can be factory made or user selectable. Proper operation of the outflow rate regulator of the present invention requires that the fluid entering the narrow fluid passages is free from solid particles of sizes that could block the narrow fluid passages of the system. A particle retainer preferably consisting in a low resistance particulate material filter must be installed between the surgical hand-piece and the outflow rate regulator device to ensure proper operation of the outflow rate regulator. Among the advantages of the present invention it can be mentioned that it is low cost, simple, effective to reduce post-occlusion surge, reliable and that it does not affect performance of the lensectomy apparatus while operating in non-occlusion conditions.

### BRIEF DESCRIPTION OF THE DRAWINGS

FIG. 1 is a schematic drawing of an ophthalmic surgical system including the outflow rate regulator of the present invention.

FIG. 2A is a detailed longitudinal view of one embodiment of an outflow rate regulator of the present invention.

FIG. 2B is a detailed cross sectional view of one embodiment of an outflow rate regulator of the present invention.

FIG. 2C is a graph depicting the pressure difference versus flow rate obtained by using the outflow rate regulator depicted in FIGS. 2A and 2B.

FIG. 3A is a detailed longitudinal view of one embodiment of an outflow rate regulator of the present invention.

FIG. 3B is a detailed cross sectional view of one embodiment of an outflow rate regulator of the present invention.

FIG. 23 is a graph depicting the pressure difference versus flow rate obtained by using the outflow rate regulator depicted in FIGS. 23 and 23.

FIG. 4A is a detailed longitudinal view of one embodiment of an outflow rate regulator of the present invention.

FIG. 4B is a detailed cross sectional view of one embodiment of an outflow rate regulator of the present invention.

FIG. 4C is a detailed longitudinal view of one embodiment of the outflow rate regulator of the present invention including an adjustment knob for a user to select a desired performance pattern.

FIG. 4D is a graph depicting the pressure difference versus flow rate obtained by using the outflow rate regulator depicted in FIGS. 4A, 4B and 4C.

FIG. 4E is a graph depicting a trans device pressure difference versus flow rate obtained by using the outflow rate regulator of the present invention and showing an hysteresis curve that is on purpose obtained by design characteristics

FIG. 5A is a detailed longitudinal view of one embodiment of an outflow rate regulator of the present invention incorporating a compression low compliance bellows.

FIG. 5B is a detailed cross sectional view of one embodiment of an outflow rate regulator of the present invention incorporating a disc shaped low compliance bellows

FIG. 5C is an enlarged cross sectional view of variable section fluid passage portion of the embodiments shown in FIGS. 5A and 5B

FIG. 6 is a graph depicting a pressure difference versus flow rate obtained by using the outflow rate regulator of the present invention depicted in FIG. 5.

FIG. 7A is a detailed cross sectional view of one embodiment of an outflow rate regulator of the present invention including a solid residue retainer and using an extension bellows.

FIG. 7B is an enlarged cross sectional view of the variable section fluid passage portion of the embodiments shown in FIG.7A.

FIG. 8A is a detailed cross sectional view of one embodiment of an outflow rate regulator of the present invention including a solid residue retainer and using a diaphragm to vary the extension of a narrow fluid passage.

FIG. 8B is an upper view at section level with label ‘b’ of the flow regulating device shown in FIG. 8A.

FIG. 9A is a lateral sectional view of an alternative embodiment incorporating a movable ball and spring.

FIG. 9B is an axial view from the access side of the embodiment depicted in FIG. 9A.

### NUMERALS FROM FIGURES

Particle retainer 10, retainer in port 12, particle retaining chamber 14, low resistance filtering membrane 16, clean fluid exit side 18, retainer out port 20, outflow rate regulator 30, regulator in port 32, regulator out port 34, diaphragm 40, calibrated permanent fluid passage 42, blocking fluid passage 44, diaphragm bed 46, access side 48, exit side 50, blockable fluid passage 52, blockable fluid passage 54, blockable fluid passage 56, slit 60, slit non blocking portion 62, diaphragm 70, calibrated bellows 76, variable area fluid passage 78, variable section flow regulator needle 80, reflux bypass 83, reflux valve 84, adjustment element 86, phacoemulsification surgical system 100 hand-piece 102, phacoemulsification needle 104, infusion bottle 106, infusion line 108, infusion sleeve 109, infusion solenoid valve 110, phacoemulsification needle 11, aspiration line 112, aspiration line sensor 114, aspiration pump 116, waste fluid outlet 117, collector bag 11 8, adjustment knob 120, console controls 122, fluid passage 130, fluid narrow channel 132, spring 140, body 142, body guides 144, septum 146, spring holder 148, walls 150, clear space 152

### DETAILED DESCRIPTION

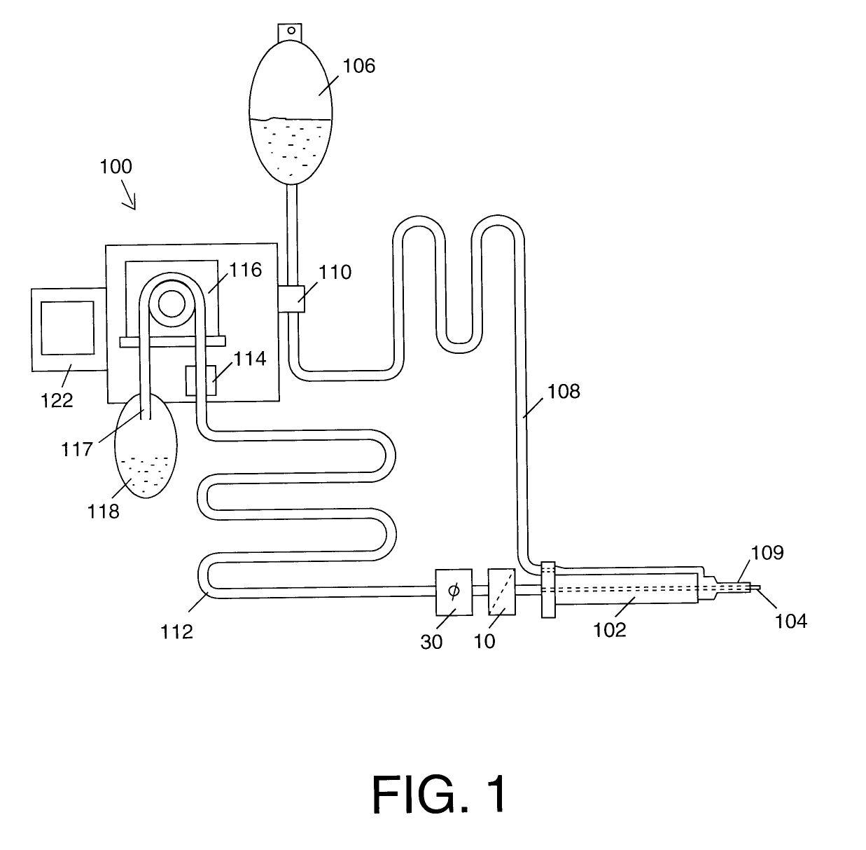

In FIG. 1, there is shown a phacoemulsification surgical system 100 in which an outflow rate regulator 30 of the present invention may be used to advantage. Surgical system 100 has an infusion bottle 106 connecting through an infusion line 108 to an infusion sleeve 109 to perfuse the anterior chamber of the eye.

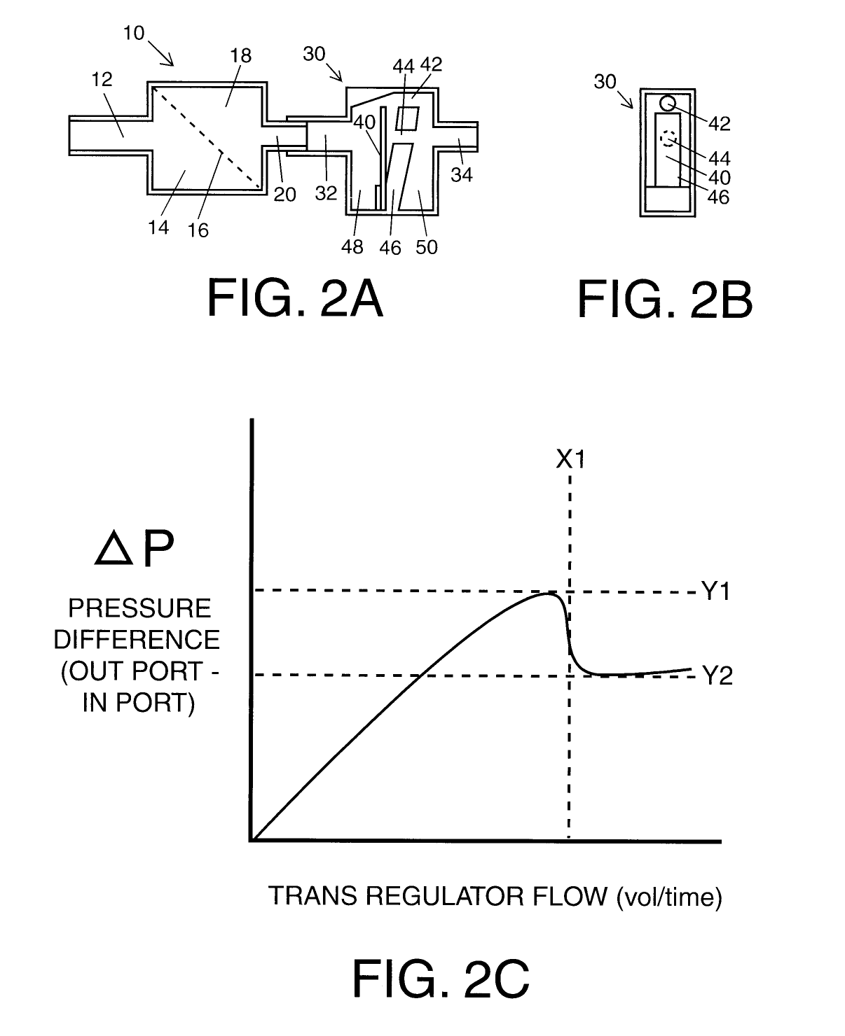

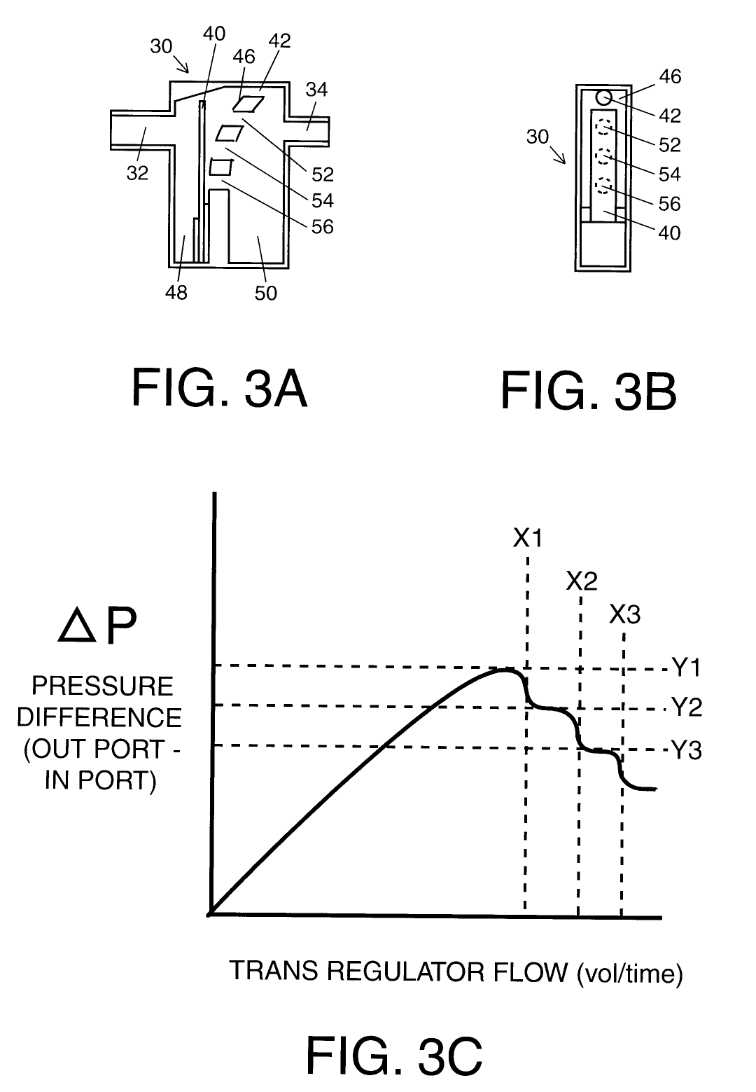

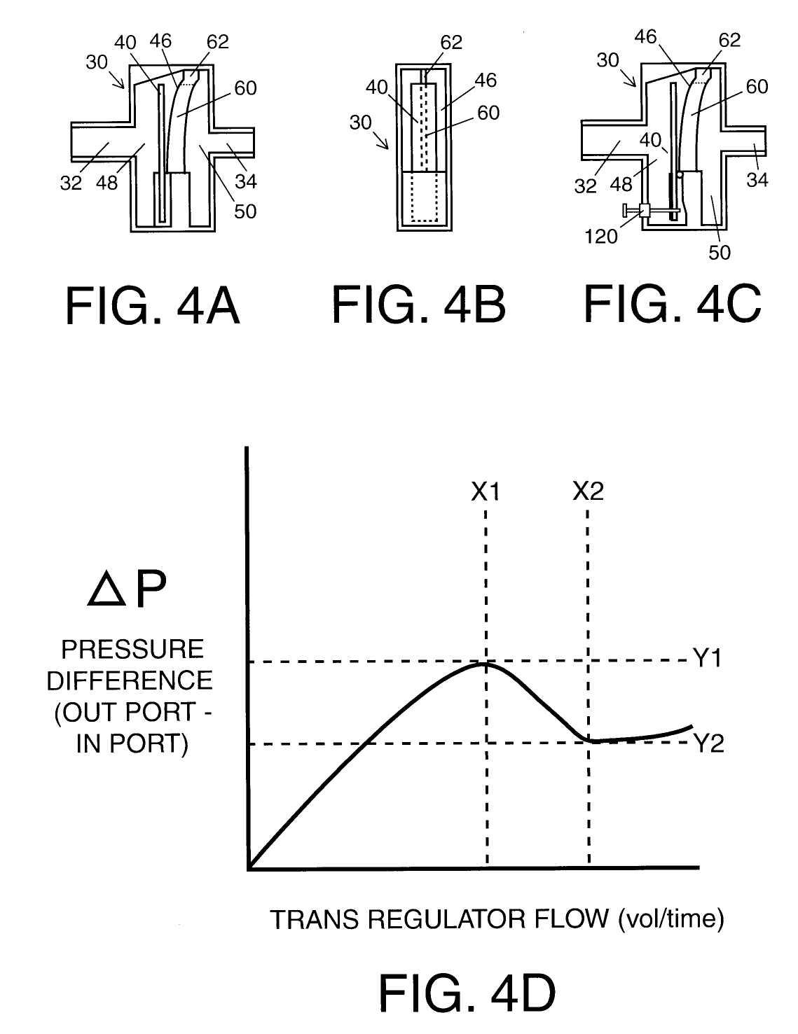

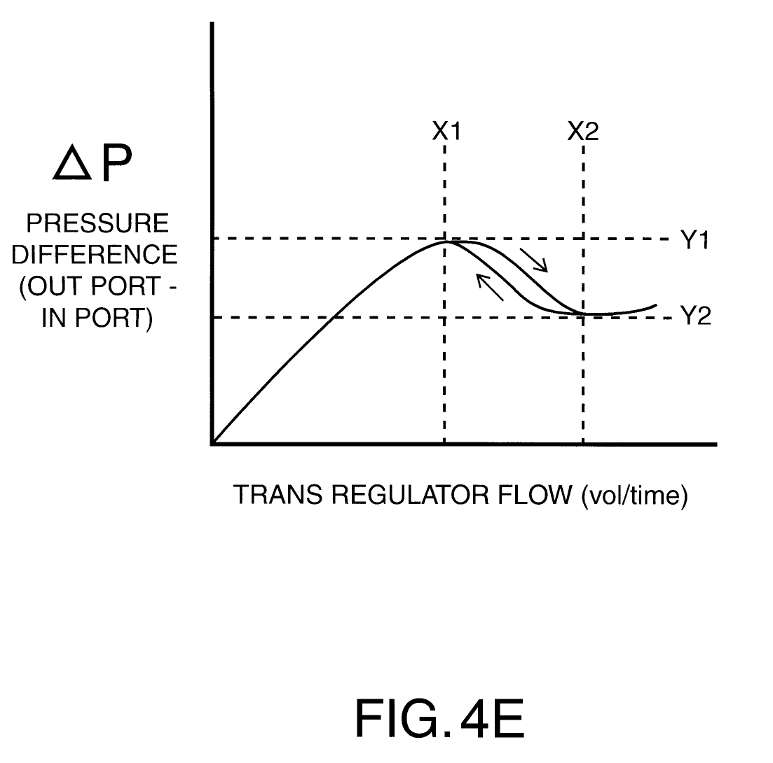

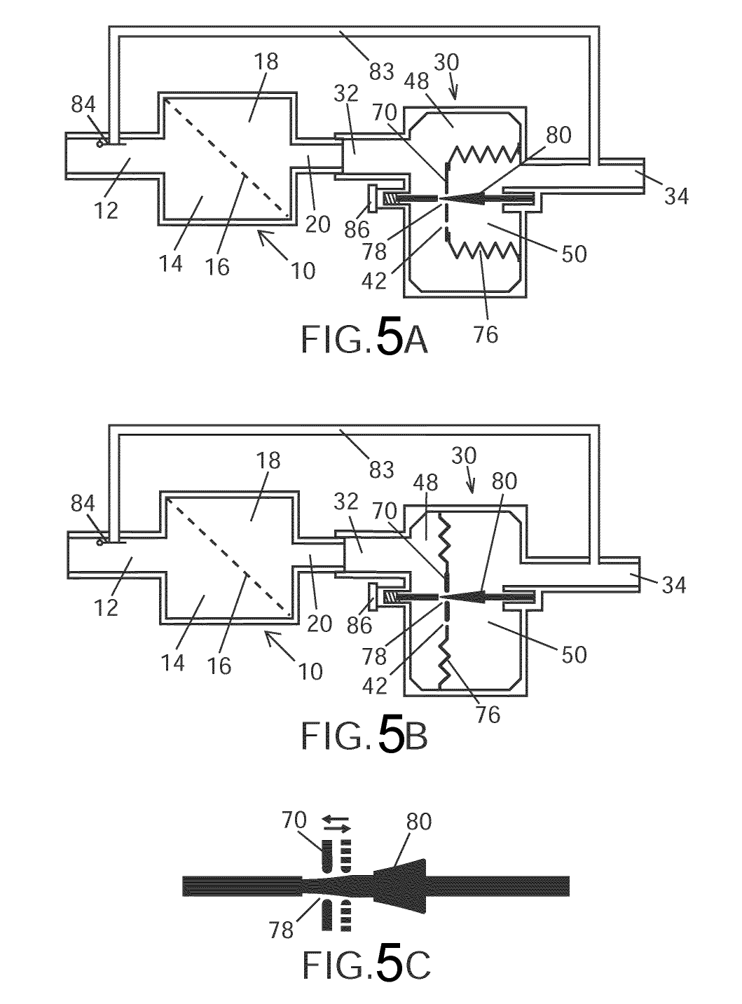

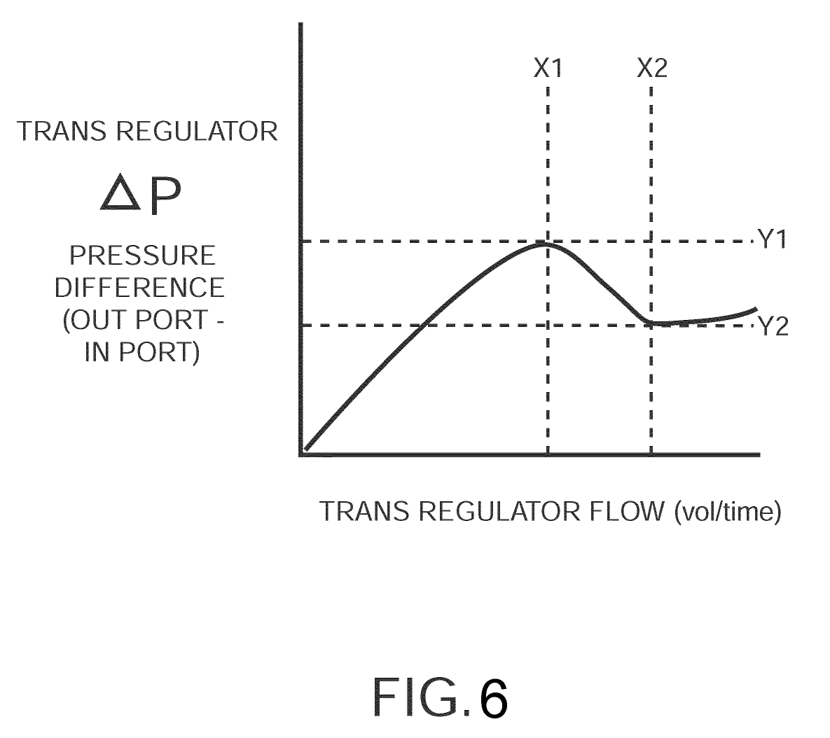

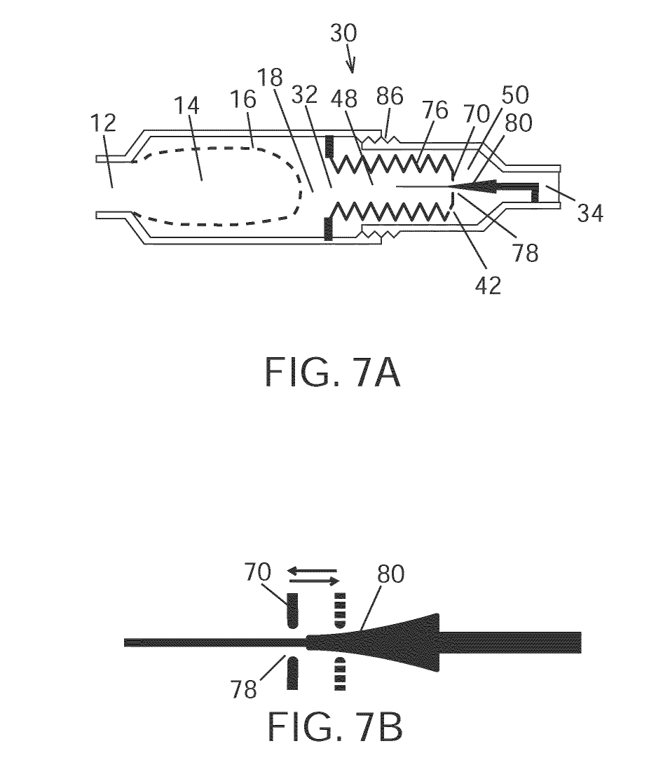

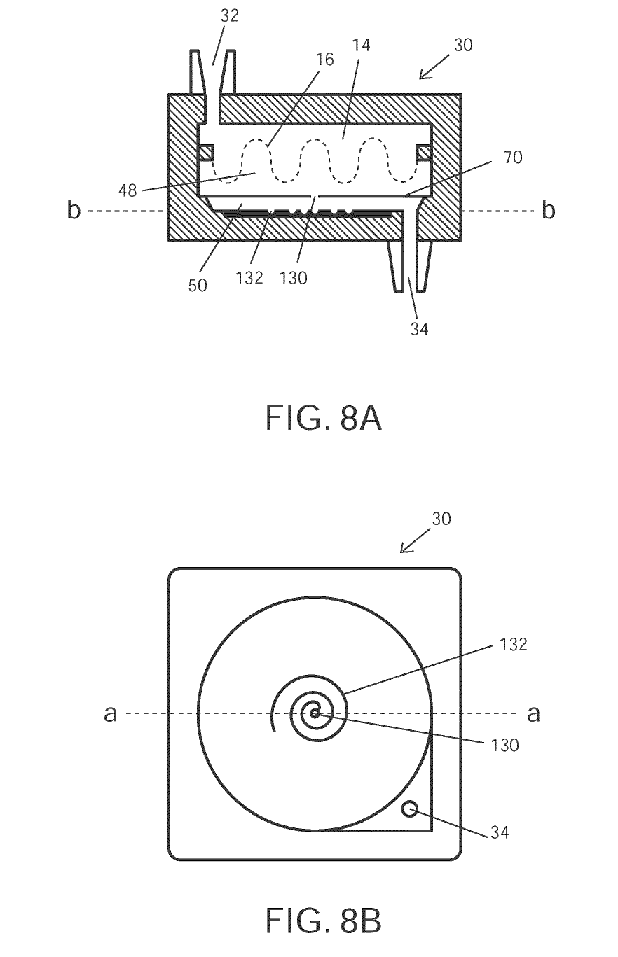

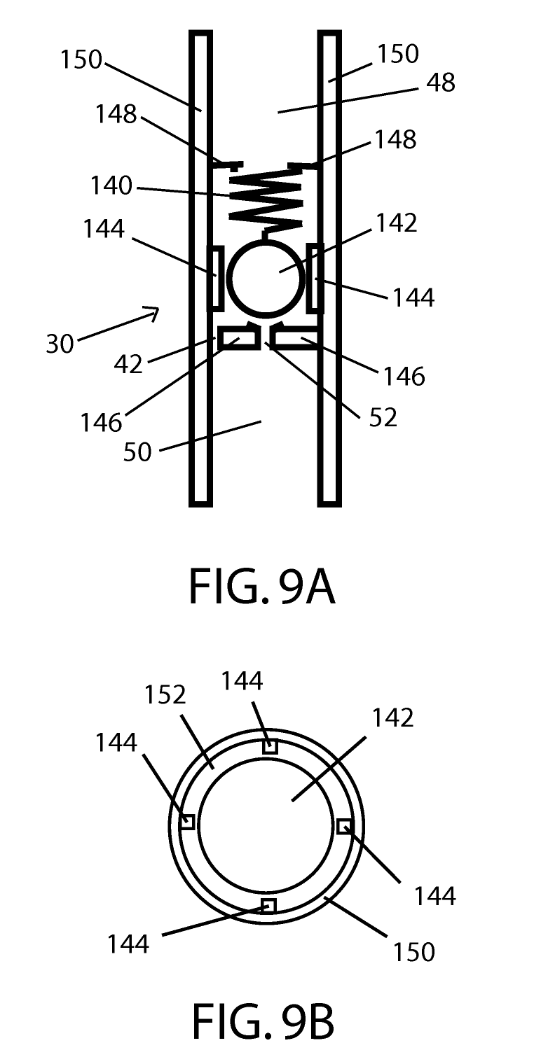

Alternatively line 108 can connect to a secondary port infusion instrument such as an anterior chamber maintainer or irrigating instrument for the same purpose. An infusion line solenoid valve 110 has a clamping action upon infusion line 108. A hollow phacoemulsification needle 104 placed at the distal end of a phacoemulsification hand-piece 102 operates with the distal end placed at the anterior chamber of the eye. Needle 104 is proximally in fluid connection with a solid particle retainer 10 which is in fluid downstream connection with outflow rate regulator 30 of the present invention. The output of outflow rate regulator 30 is in fluid connection with an aspiration line 112 connecting downstream to an aspiration pump 116 having a waste fluid outlet 117. Waste fluid outlet connects to a waste fluid collector bag 118. A set of controls 122 allows an operator to program and activate surgical system 100. An outflow rate regulator system in accordance with the present invention generally includes a flow rate regulator device 30. It is desirable for proper operation of the flow regulator device 30 that fluid passing through the flow rate regulator device 30 is free of solid material above a critical particle size preferably 50 microns. As shown in FIG. 2A, FIG. 5A and FIG. 5B particle retainer 10 is always provided that is mainly composed of a retainer input port 12 opening to a particle retaining chamber 14. A low insertion resistance filtering membrane 16 is placed fully across the fluid path of particle retainer 10. A clean fluid exit side 18 directs the filtered fluid to retainer output port 20. One embodiment of flow regulator device 30 shown in FIGS. 2A and 2B is composed of a regulator input port 32 communicating with an access side 48. An exit side 50 conducts the exiting fluid to output port 34. A movable diaphragm 40 is disposed to progressively displace towards a diaphragm bed 46 occluding a blocking fluid passage 44 when deforming or displacing in response to a pressure difference between access side 48 and exit side 50. A non-blocking fluid passage 42 is placed between chambers 48 y 50 and is designed to maintain the device permanently patent to fluid flow avoiding latch-up. Another embodiment of flow regulator device 30 is shown in FIGS. 3A and 3B composed of a regulator input port 32 communicating with an access side 48. An exit side 50 conducts the exiting fluid to output port 34. A flexible diaphragm 40 is disposed to progressively displace towards a diaphragm bed 46 occluding in sequence a series of fluid passages 52, 54, 56 when bending by the action of a pressure difference between access side 48 and exit side 50. A non-blocking fluid passage 42 is placed between chambers 48 y 50 and designed to maintain permanently patent to fluid flow avoiding latch-up. One preferred embodiment of flow regulator device 30 shown in FIGS. 4A and 4B and is composed of a regulator input port 32 communicating with an access side 48. An exit side 50 conducts the exiting fluid to output port 34. A flexible membrane is disposed o progressively displace towards a membrane bed progressively occluding slit shaped fluid passage 60 when bending by the action of a pressure difference between access side 48 and exit side 50. A non-blocking portion 62 of the fluid passage is unreachable to membrane this portion maintaining permanently patent to fluid flow. FIG. 4C depicts a variation in design that further included s adjustment knob 120 providing the manufacturer or a user means to vary the angle between diaphragm 40 and diaphragm bed 46 being this one method to adjust the dynamic response curve for flow regulator device 30 to a desired pattern according o surgical conditions. Another possible embodiment of a flow regulator device is shown in FIGS. 5A, 5B and 5C and s and composed of a regulator input port 32 communicating with an access side 48. An exit side 50 conducts the exiting fluid to output port 34 toward the aspiration pump. A diaphragm 70 is attached to a calibrated deformable bellows 76, both elements separating access side 48 and exit side 50. Diaphragm 70 has a calibrated opening that in combination with an axially disposed variable section needle 80 constitutes a variable section fluid passage 78 between chambers 48 and 50. Needle 80 is usually cone shaped with the wider portion oriented towards the side of chamber 50. As an option a secondary calibrated opening 42 can be included to prevent latch up. Also as an option an adjustment element 86 can be included to regulate the response curve. In one configuration shown in FIG. 5A diaphragm 70 is mounted over a calibrated compression bellows. Alternatively, as shown in FIG. 5B diaphragm 70 is mounted over a calibrated disk shaped bellows. Also as an option an adjustment element 86 can be included. Optionally a calibrated spring can be added to support the diaphragm from either side to alter the pressure versus flow response curve of the device in a favourable manner (not shown). FIGS. 5A and 5B incorporate a reflow duct 83 and reflow valve 84 operable during reflow conditions to avoid waste fluid to deliver lens particles back to the eye chambers. As shown in FIG. 5C, variable section needle 80 is disposed to centrally cross in a perpendicular direction the calibrated opening of diaphragm 70 with the wider section towards chamber 50. The variation of the section of needle 80 along its main axis is designed to provide a desired performance curve when operating in combination with the calibrated perforation of diaphragm 70 determining a fluid passage 78 of variable area. Variation in fluid passage area 78 occurs by relative displacement of diaphragm 70 and its calibrated opening along the variable section fixed needle 80. FIGS. 7A and 7B illustrate one preferred embodiment that incorporates solid particle retainer system 10 to the body of an outflow rate regulator 30. Added features are the optional adjustment feature provided by adjustment element 86 operable to modify the resting relative position of diaphragm 70 and its calibrated opening over fixed variable section needle 80.v FIGS. 8A and 8B illustrate another embodiment that incorporates a solid particle retainer membrane 14 within an outflow rate regulator device 30. A diaphragm 70 is operable to displace towards a flat bed with a calibrated fluid channel 132 as a function of the pressure difference between an access side 48 and an exit side 50. A fluid passage 130 communicates access side 48 and exit side 50 in a way that when contact occurs between diaphragm 70 and the flat bed, fluid passage 130 delivers fluid to narrow fluid channel 132.

OPERATION: Infusion bottle 106 provides pressurized inflow fluid by gravitational or other forces to infusion line 108. Solenoid valve 110 opens and closes inflow to the eye by clamping infusion line 108 on console command. Infusion line 108 is in fluid communication with the anterior chamber of the eye through infusion sleeve 109 or other infusing devices providing pressurized fluid to the anterior chamber of the eye. Aspiration pump 1 6 produces a vacuum in aspiration line 12 that is transmitted upstream to hollow phacoemulsification needle 104 tip. A fluid outflow and a vacuum at the tip of needle 104 removes lens fragments. Lens fragments are retained by particle retainer 10 to avoid clogging the narrow fluid passages downstream. The filtered fluid travels across outflow rate regulator 30 and is conducted through aspiration line 112 to aspiration pump 16. Aspiration pump 16 delivers the waste fluid to a waste fluid outlet 117 and is collected by waste fluid collector bag 118. During unobstructed operation of the phacoemulsification system, aspiration line 112 vacuum remains relatively low and the actual outflow rate can increase almost linearly with the console preset flow rate. In a standard system, upon occlusion of phacoemulsification needle 104 by lens material, aspiration line 112 vacuum increases by the sustained action of aspiration pump 116 partially collapsing aspiration tubing 112 and expanding bubbles eventually present in the aspiration ducts. After an occlusion breaks, fluid rapidly exits the anterior chamber into the aspiration line and a peak of outflow rate is observed through hollow needle 104 to fill the fluid void produced by the expansion of the partially collapsed tubing 112 and contracting bubbles. This peak of fluid outflow is known as post-occlusion surge and can collapse the anterior chamber of the eye and promote complications. The incorporation of the outflow rate regulator 30 of the present invention allows to significantly reduce the post-occlusion surge even when operating at the very high vacuum levels (i.e. above 600 mmHg) available in the most modern phacoemulsification systems available today. Operation of all embodiments depicted in FIGS. 2, 3, and 4 consider displacement of a flexible membrane or diaphragm 40 progressively occluding one or more fluid passages between an access side 48 and an exit side 50. In this way, flow across the outflow rate regulator is incrementally restricted according to the pressure gradient across rate regulator 30 access and exit sides. As the pressure gradient is reduced, the occluded fluid passages reopen allowing higher flow rates. A calibrated permanent fluid passage 42 permits a controlled equilibration of the pressure difference minimizing the surge phenomenon and avoiding latch up of the displacing membrane or diaphragm. As the pressure gradient drops, diaphragm 40 returns to incrementally less occluding positions restoring operation at normal flow rate with low pressure differences across device 30. Preferred embodiment depicted in FIGS. 4A, 4B and 4C is designed to provide a graded response. Incremental deformation of flexible or movable membrane or diaphragm 40 produces incremental occlusion of fluid passage 60 in a selected pattern determined by membrane 40 elastic properties, architecture, membrane bed 46 shapes, membrane 40 relative position, access side 48 three-dimensional architecture among others. Graphs depicted in FIGS. 2C, 3C, 4D and 4E illustrate possible pressure gradient versus flow rate curves corresponding respectively to the outflow rate regulator embodiments shown in FIGS. 2, 3 and 4. Preferred embodiment depicted in FIGS. 5 and FIGS. 7 are designed to provide a graded response to post-occlusion surge. During occlusion conditions the pressure gradient between chambers 48 and 50 is near zero. During normal, non-occlusion operation conditions a pressure gradient appears between chambers 48 and 50 that may displace to some extent diaphragm 70 and its calibrated opening crossed by needle 80. This displacement occurs along the axis of needle 80 in a zone where needle 80 section is designed not necessarily to contribute to increase resistance to flow. When occlusion breaks with high vacuum in the aspiration line, the pressure difference between access and exit sides 48 and 50 steeply increases producing a proportional displacement of diaphragm 70 with calibrated opening into a wider section of needle 80 narrowing the fluid passage 78 in a way that resistance to fluid flow between chambers 48 and 50 increases. In this manner net flow is limited reducing the rate of fluid extraction from the anterior chamber and avoiding anterior chamber collapse after the occlusion break. With device 30 in operation, the post-occlusion break peak outflow is clamped to moderate flow rates (i.e. <60 cc/min) allowing fluid from infusion line 108 to timely refill the eye chambers preventing collapse. As fluid traverses through the transiently increased resistance between chambers 48 and 50, the pressure difference reduces allowing the moving parts return to their pre-surge position, increasing in the area of variable fluid passage 78, returning to non-occlusion normal flow rate operation conditions. The graph depicted in FIG. 6 illustrates a typical pressure gradient versus flow rate curve corresponding to the performance of the outflow rate regulator 30 embodiment shown in FIG. 5 and FIG. 7. FIGS. 2C, 3C, 4D, and 5C include ruler markings X1, X2, Y1, Y2 that allow a better description of the pressure gradient versus flow rate curve of outflow rate regulators of the present invention.

As can be interpreted from the graphs shown, flow rate across outflow rate regulator devices 30 of the present invention will increase almost linearly with the pressure gradient when in non-occlusion operation up to a desirable level typically about 40 to 60 ml/min. When the pressure gradient across device 30 exceeds a preset value, the fluid passage will progressively narrow increasing resistance and reducing the flow rate. In this way post-occlusion surge is prevented.

Alternative embodiment depicted in FIGS. 8A and 8B operates by varying the resistance to flow by the action of a diaphragm 70 that progressively contacts a flat bed with a narrow fluid channel 132 in a way that an increasing pressure difference between an access side 48 and an exit side 50 produces an increasing contact zone increasing the effective length of narrow channel 132 increasing resistance to flow. An important design aspect is to produce the modifications in the fluid passage section with minimal volume compliance, to obtain fast responses to variations in pressure differences.

Similarly, embodiment shown in FIGS. 8A and 8B is designed to produce an increase in effective length of narrow channel 132 as a function of pressure differences across diaphragm 70 with minimal volume compliance, to obtain fast variations in flow resistance in response to variations in pressure differences.

The embodiment shown in FIGS. 9A and 9B includes a spring 140 holding a movable body 142 suspended between guides 144 and leaving a clear space 152 for free fluid flow. Septum 146 incorporates permanent fluid passage 42 and blockable fluid passage 52. A spring holder rim 148 houses the fixed end of spring 140. The complete system is enclosed by wall 150 having a diameter between 3 and 6 millimetre comparable to standard aspiration line diameters. During operation, normal flow rates (i.e. below 50 cc/min) maintain body 142 at sufficient distance from blockable passage 52 opening.

Depending on design characteristics of an outflow rate regulator 30, the pressure gradient versus flow rate curve for a particular device 30 can vary in several ways determining different thresholds, inflections and slopes of the flow versus pressure gradient curve.

Also depending on variations in design, a different curve can be traced when plotting while moving from a low to high vacuum difference and when plotting moving from a high to low vacuum difference, a phenomenon known as hysteresis and that can be used with advantage upon design.

Dynamic behaviour can be adjusted by design in a way that different curves can be traced for a single device 30 depending on the rate of change of the pressure gradient across the device.

It will be understood for those skilled in the art that this description contains many specific details relevant only to the described embodiments. Other embodiments can be construed following the same principles of operation without departing from the present invention. For example the moving part of the variable area fluid passage 78 can be the variable section needle 80 with the diaphragm remaining fixed. The movable portion can be ball shaped. A spring can be part of the deformable portion to adjust the response curve. The permanent fluid passage can be a non-blockable portion of a bigger, partially blockable fluid passage.

Manufacturing of the present invention can be made using traditional construction techniques and/or micromachining technologies.

1. An outflow rate regulator system for a lens removing apparatus comprising:
an access side,
an exit side,
at least one fluid passage between said access side and said exit side,
a fluid channel blocking portion movable or deformable by fluid pressure difference along said access side and exit side,
wherein said fluid channel blocking portion reacts to said fluid pressure difference producing a reduction of the effective area of said fluid passage as a direct function of said fluid pressure difference.

2. The outflow rate regulator system of claim 1 further including a solid particle retaining filter at said access side.

3. The outflow rate regulator system of claim 1 further including a permanently patent fluid passage portion with a fixed area ranging between 0.008 and 0.2 square mm.

4. The outflow rate regulator system of claim 1 further including a variable area fluid passage portion with the area being adjustable in the range of 0.03 and 3 square mm by the action of said fluid blocking portion.

5. The outflow rate regulator system of claim 1 adjusted to block flow rates above a selected flow rate level.

6. Said flow rate level of claim 5 in the range between 30 to 80 cc/min.

7. The outflow rate regulator system of claim 1 wherein an increase in resistance to flow is produced by the relative displacement of a diaphragm narrowing a fluid passage.

8. The flow limiting device of claim 1 wherein an increase in resistance to flow is produced by relative displacement of a diaphragm modifying the effective length of a narrow fluid channel.

9. An outflow rate regulator system for a lens removing apparatus comprising:
an access side,
an exit side,
at least one fluid passage between said access side and said exit side,
a diaphragm,
wherein said diaphragm proportionally deforms in reaction to the fluid pressure difference between said access side and exit side producing a increase in the length of said fluid passage as a direct function of said fluid pressure difference.
